(19) United States
(12) Patent Application Publication
(10) Pub. No.: US 2006/0122587 A1
(43) Pub. Date: Jun. 8, 2006

(54) Apparatus for real time evaluation of tissue ablation

(76) Inventor: Shiva Sharareh, Laguna Niguel, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/281,179

(22) Filed: Nov. 17, 2005

Related U.S. Application Data
(60) Provisional application No. 60/629,166, filed on Nov. 17, 2004.

(51) Int. Cl. A61B 18/18

(52) U.S. Cl. 606/11; 606/7; 606/15

(57) ABSTRACT

An apparatus for the evaluation of tissue ablation is provided. The apparatus comprises a broadband (white; multiple wavelengths) light and/or laser light (single wavelength) illumination source that delivers light to the site where a lesion is being formed. Scattered light is collected from the ablated tissue and evaluated to obtain qualitative information regarding the newly formed lesion. The apparatus allows assessment of such parameters as, for example, catheter- tissue proximity, lesion formation, depth of penetration of the lesion, cross-sectional area of the lesion in the tissue, formation of char during the ablation, recognition of char from non-charred tissue, formation of coagulum around the ablation site, differentiation of coagulated from non-coagulated blood, differentiation of ablated from healthy tissue, and recognition of steam formation in the tissue for prevention of steam pop. These assessments are accomplished by measuring the intensity and spectrum of diffusely reflected light at one or more wavelengths.

### CROSS REFERENCE TO RELATED APPLICATIONS

This application claims priority from U.S. Provisional Patent Application No. 60/629,166 filed on Nov. 17, 2004 for Fiber-Optic Evaluation of Cardiac Tissue Ablation & Optical Spectroscopy.

### FIELD OF THE INVENTION

The present invention relates generally to the field of tissue ablation. More specifically, the present invention relates to a system and method for tracking and evaluating an ablation as it is formed in the human body.

### BACKGROUND OF THE INVENTION

For certain types of minimally invasive medical procedures, real time information regarding the condition of the treatment site within the body is unavailable. This lack of information inhibits the clinician when employing a medical device to perform a procedure. An example of such procedures is tumor and disease treatment in the liver and prostate. Yet another example of such a procedures is surgical ablation used to treat atrial fibrillation. This condition in the heart causes abnormal electrical signals, known as cardiac arrhythmias, to be generated in the endocardial tissue resulting in irregular beating of the heart.

The most frequent cause of cardiac arrhythmias is an abnormal routing of electricity through the cardiac tissue. In general, most arrhythmias are treated by ablating suspected centers of this electrical misfiring, thereby causing these centers to become inactive. Successful treatment, then, depends on the location of the ablation within the heart as well as the lesion itself. For example, when treating atrial fibrillation, an ablation catheter is maneuvered into the right or left atrium where it is used to create elongated ablation lesions in the heart. These lesions are intended to stop the irregular beating of the heart by creating non-conductive barriers between regions of the atria that halt passage through the heart of the abnormal electrical activity.

The lesion must be created such that electrical conductivity is halted in the localized region (transmurality), but care must be taken to prevent ablating adjacent tissues. Furthermore, the ablation process can also cause undesirable charring of the tissue and localized coagulation, and can generate evaporate water in the blood and tissue leading to steam pops.

Currently, lesions are evaluated following the ablation procedure, by positioning a mapping catheter in the heart where it is used to measure the electrical activity within the atria. This permits the physician to evaluate the newly formed lesions and determine whether they will function to halt conductivity. If it is determined that the lesions were not adequately formed, then additional lesions can be created to further form a line of block against passage of abnormal currents. Clearly, post ablation evaluation is undesirable since correction requires additional medical procedures. Thus, it would be more desirable to evaluate the lesion as it is being formed in the tissue.

A known method for evaluating lesions as they are formed is to measure electrical impedance. Biochemical differences between ablated and normal tissue can result in changes in electrical impedance between the tissue types. Although impedance is routinely monitored during electrophysiologic therapy, however, it is not directly related to lesion formation. Measuring impedance merely provides data as to the location of the tissue lesion but does not give qualitative data to evaluate the effectiveness of the lesion.

Another approach is to measure the electrical conductance between two points of tissue. This process, known as lesion pacing, can also determine the effectiveness of lesion therapy. This technique, however measures only the success or lack thereof from each lesion, and yields no real-time information about the lesion formation.

Thus, there is a need for an instrument capable of measuring lesion formation in real-time, as well as detect the formation of charred tissue and coagulated blood around the ablation catheter.

### SUMMARY OF THE INVENTION

According to the invention, an apparatus and method for the evaluation of tissue ablation is provided. The apparatus comprises a broadband (white; multiple wavelengths) light and/or laser light (single wavelength) illumination source that delivers light to the site where a lesion is being formed. Reflected light is collected from the ablated tissue and evaluated to obtain qualitative information regarding the newly formed lesion.

The apparatus allows assessment of such parameters as, for example, lesion formation, depth of penetration of the lesion, cross-sectional area of the lesion in the tissue, formation of char during the ablation, recognition of char from non-charred tissue, formation of coagulum around the ablation site, differentiation of coagulated from non-coagulated blood, differentiation of ablated from healthy tissue, tissue proximity, and recognition of steam formation in the tissue for prevention of steam pop. These assessments are accomplished by measuring the intensity and spectrum of diffusely reflected light at one or more wavelengths

In general, ablation systems comprise an ablation catheter or similar probe having an energy-emitting element. The energy-emitting element delivers energy forming a lesion in the targeted tissue. Typical elements comprise a microwave ablation element, a cryogenic ablation element, a thermal ablation element, a light-emitting ablation element, an ultrasound transducer, and a radio frequency ablation element. The ablation catheter may be adapted to form a variety of lesions such as linear lesions or a circumferential lesion. The element is connected to an energy source that can be varied to control the formation of the lesion. For example, providing higher current to an electrical coil ablation element will cause a deeper lesion and may result in increased steam pops and/or charring of neighboring tissue.

In the present invention, the ablation catheter is modified to include a light emitter that provides broadband and/or laser light to the lesion site. The emitter may comprise a fiber optic cable or a laser mounted within the tip of the ablation catheter. A light detector is also mounted on the ablation catheter to collect diffusely scattered illumination light. Collection optics in the ablation catheter may utilize lenses, mirrors, gratings, optical fibers, liquid or hollow waveguides, or any combination thereof to transmit the diffusely scattered light to a detection system. The detection system comprises a wavelength selective element such as a spectrograph(s) that disperses the collected light into constituent wavelengths, and a device that quantifies the light. The quantification device may comprise a charged coupled device (CCD) that simultaneously detects and quantifies light intensities. Alternatively, a number of different light sensors, including photodiodes, photomultipliers or complementary metal oxide semiconductor (CMOS) detectors may be use in place of the CCD converter.

The CCD converts these measured light intensities into an electrical signal that can be processed with a computer and displayed graphically to the end-user of the ablation device. During surgical ablation, the operator obtains information about the lesion as it is being formed or detects lesions that have already been formed. For example, the intensity of the scattered light changes due to ablation of tissue allowing for an existing lesion to be located as the ablation catheter is advanced over tissue. Moreover, the depth of the lesion causes a corresponding change in the spectrum of scattered light. The operator can use this information to increase or decrease the energy delivered to the site varying the depth of the lesion.

### BRIEF DESCRIPTION OF THE DRAWINGS

The features and advantages of the invention will be apparent to those of ordinary skill in the art from the following detailed description of which:

FIG. 1 is a schematic drawing showing the components of the ablation evaluation device of the present invention.

FIG. 2 is a front side view cutaway view of an example of an ablation catheter modified with the light emission and detection configuration of the present invention.

FIG. 3 is a rear side view of an ablation catheter modified with the light emission and detection configuration of the present invention.

FIG. 4 is a schematic view of a variation of the catheter positioning system of the present invention in situ.

### DETAILED DESCRIPTION OF THE PREFERRED EMBODIMENTS

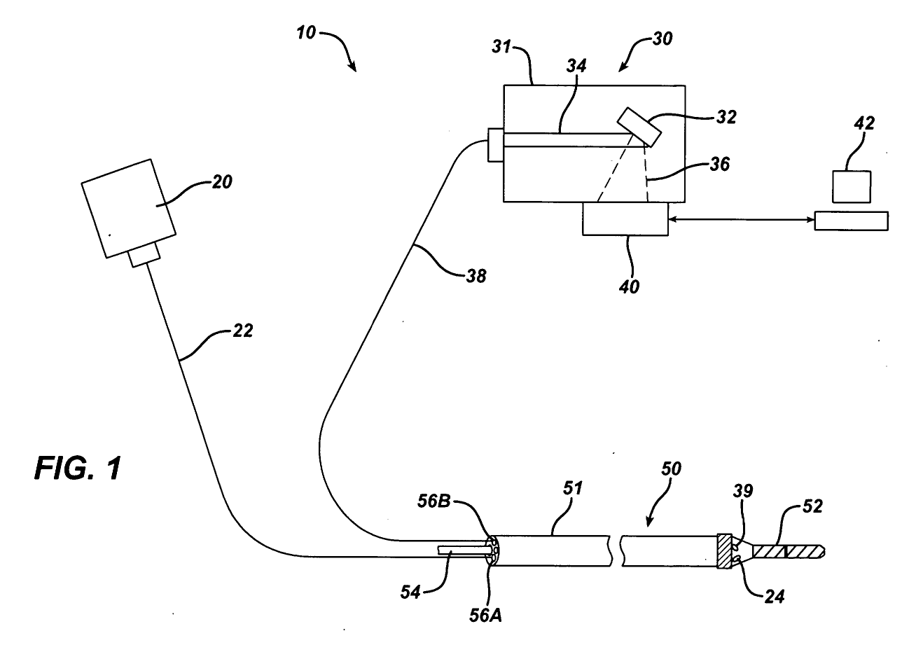

An apparatus for evaluating tissue during surgical ablation will be described with reference to FIGS. 1-4. As shown in FIG. 1, the apparatus generally comprises a surgical ablation catheter 50 which may be used in any region of the body where ablation procedures are performed such as the heart, liver or prostrate. Ablation catheter 50 generally comprises an elongate body 51 having an ablation element 52 located at its distal end. A guidewire 54 may extend from the proximal to the distal end of the elongate body 51. As will be described below, the guidewire 54 may be employed to position the catheter 50 at the location where ablation of tissue is to occur. Alternatively and preferably, the ablation catheter 50 is steerable and will not require a guidewire to position the ablation catheter at the site where the lesion is to be formed. As is described below, ablation element 52 emits energy that causes a lesion to be formed in tissue

According to the present invention, ablation catheter 50 is modified to have at least one emitting device 24 and collection device 39 mounted at its distal end. The catheter also includes at least two lumens 56A and 56B that permit passage of optical cables 22 and 38 from the proximal end of catheter 50 to emitting device 24 and collection device 39 respectively. The device 24 emits a bandwidth of electromagnetic energy and may comprise, for example, a fiber optic cable, LED or laser mounted at or near the distal end of the ablation catheter. The collector 39 mounted in the ablation catheter directs a bandwidth of scattered electromagnetic light to detection component 30. Collection device 50 may comprise lenses, mirrors, gratings, optical fibers, liquid or hollow waveguides, or any combination thereof to transmit the diffusely scattered light to a detection system.

Alternatively, the light emitting device 24 and collection device 39 may be a mounted in a separate catheter or may comprise fiber optic cables mounted externally of the ablation catheter 50. In this configuration the external emitting and collection devices are located in proximity to the distal end of catheter 50 illuminating either an existing lesion, or a lesion as it is being formed, with a bandwidth of electromagnetic energy and collecting scattered electromagnetic energy from the lesion and surrounding tissue.

A light source 20 supplies a broadband (white; multiple wavelengths) light and/or laser light (single wavelength) illumination to device 24 via cable 22. The light is projected into the surrounding tissue where it is scattered. The collection device 39 collects the scattered light and transmits it, via optical cable 38, to a detection component 30. Detection component 30 may comprise, for example, a wavelength selective element 31 that disperses the collected light into constituent wavelengths, and a quantification apparatus 40. The at least one wavelength selective element 31 includes optics 32, as are known in the art, for example a system of lenses, mirrors and/or prisms, for receiving incident light 34 and breaking it into desired components 36 that are transmitted into quantification apparatus 40.

Quantification apparatus 40 translates measured light intensities into an electrical signal that can be processed with a computer 42 and displayed graphically to the end-user of the ablation device. Quantification apparatus 40 may comprise a charged coupled device (CCD) for simultaneous detection and quantification of these light intensities. Alternatively, a number of different light sensors, including photodiodes, photomultipliers or complementary metal oxide semiconductor (CMOS) detectors may be use in place of the CCD converter. Information is transmitted from the quantification device 40 to a computer 42 where a graphical display or other information is generated regarding parameters of the lesion such as lesion formation, depth of penetration of the lesion, cross-sectional area of the lesion in the tissue, formation of char during the ablation, recognition of char from non-charred tissue, formation of coagulum around the ablation site, differentiation of coagulated from non-coagulated blood, differentiation of ablated from healthy tissue, and recognition of steam formation in the tissue for prevention of steam pop.

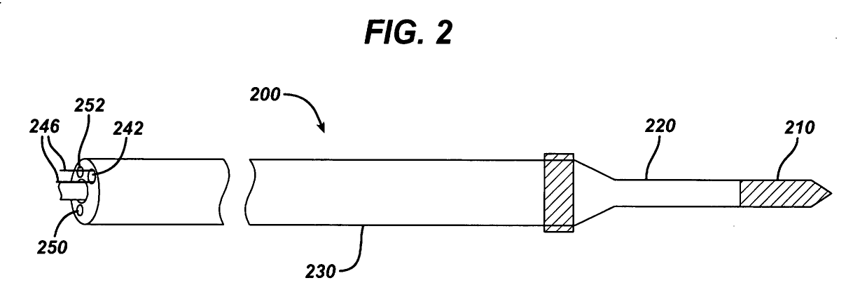

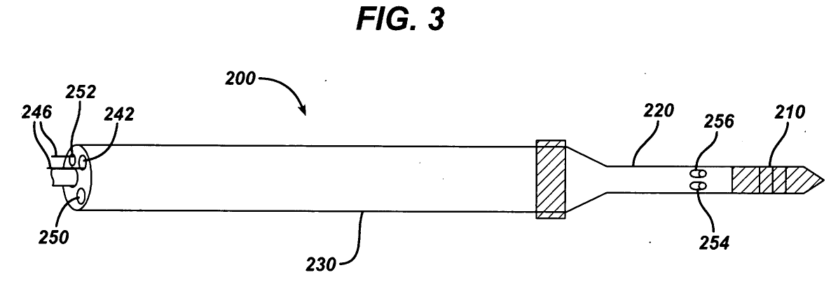

Another example of an ablation device modified in accordance with the present invention is shown in FIGS. 2-3. As shown in FIG. 2, an ablation element 210 is located along the distal end portion 220 of the steerable catheter shaft 230. Catheter shaft 230 is preferably an elongated, substantially tubular flexible body that is capable of navigating a body lumen. The shaft 230 includes electrical lumen 242 and fiber optic lumens 250 and 252. The catheter shaft 230 is placed within the body and steered to the desired point where tissue ablation is to occur such that actuating the ablation element 210 when the causes the formation of a lesion in the target tissue.

As shown in FIG. 3, an LED 254 and light detector 256 are mounted in the catheter shaft 230 proximal to the ablation element 210. The LED 254 and light detector 256 communicate with light source 20 and detection component 30 via optical cables passing through lumens 250 and 252 respectively. As a lesion is being formed by the emission of energy from the ablation element 210 the LED 254 emits light that is scattered by the ablated tissue, gathered by light detector 256 and communicated back to detection component 30.

Although described above with reference to the ablation devices described above, the present invention may be employed with a wide variety of surgical ablation devices. Exemplary variations of surgical ablation devices are described in U.S. Pat. No. 6,522,930 the disclosure of which is incorporated by reference. The ablation assembly described therein includes an ablation member that is attached to a delivery member in order to access and position the ablation member at the site of the target tissue. The delivery member may take the form of an over-the-wires catheter, wherein the “wires” include first and second guidewires. Preferably, the first guidewire is a balloon anchor wire or a deflectable guidewire. Alternatively, the wires may be engaged by external tracking sleeves. The delivery member comprises an elongated body with proximal and distal end portions. The elongated body preferably includes a first guidewire lumen, a second guidewire lumen, and an electrical lead lumen.

Each lumen extends between a proximal port and a respective distal end. The distal ends of the lumens extend through the ablation member, as described in greater detail below. Although the wire, fluid and electrical lead lumens may assume a side-by-side relationship, the elongated body can also be constructed with one or more of these lumens arranged in a coaxial relationship, or in any of a wide variety of configurations that will be readily apparent to one of ordinary skill in the art.

The elongated body of the delivery member and the distally positioned ablation member desirably are adapted to be introduced into an atrium, preferably through the transeptal sheath. Therefore, the distal end portion of the elongated body and the ablation member are sufficiently flexible and adapted to track over and along the guidewires positioned within the left atrium, and more preferably seated within two of the pulmonary veins that communicate with the left atrium.

The elongated body comprises an outer tubular member that preferably houses electrical lead tubing, fluid tubing, first guidewire tubing and second guidewire tubing. Each of the tubing extends at least from the proximal end portion of the elongated body to the distal end portion, and at least partially through the ablation member, as described below. The tubing's are arranged in a side-by-side arrangement; however, as noted above, one or more of the tubing can be arranged in a coaxial arrangement. Moreover, one or both of the wire tracking means could be located outside of the tubular member, as tubular sleeves.

Notwithstanding the specific delivery device constructions just described, other delivery mechanisms for delivering the ablation member to a desired ablation region are also contemplated. For example, while an “over-the-wire” catheter construction was described, other guidewire tracking designs may also be suitable substitutes, such as for example catheter devices known as “rapid exchange” or “monorail” variations wherein the guidewire is only housed within a lumen of the catheter in the distal regions of the catheter. In another example, a deflectable tip design may also be a suitable substitute. The latter variation can also include a pullwire which is adapted to deflect the catheter tip by applying tension along varied stiffness transitions along the catheter's length, as described above.

The proximal end portion of the elongated body terminates in a coupler. In general, any of several known designs for the coupler would be suitable for use with the present tissue ablation device assembly, as would be apparent to one of ordinary skill. For example, a proximal coupler may engage the proximal end portion of the elongated body of the delivery member. The coupler includes an electrical connector that electrically couples one or more conductor leads, which stem from the ablation member and extend through the electrical lead tube, with an ablation actuator. The coupler also desirably includes another electrical connector that electrically couples one or more temperature sensor signal wires to a controller of the ablation actuator.

The ablation member has a generally tubular shape and includes an ablation element. The ablation element may include a variety of specific structures adapted to deliver energy sufficient to ablate a defined region of tissue. Suitable ablation elements for use in the present invention may therefore include, for example, but without limitation: an electrode element adapted to couple to a direct current (“DC”) or alternating current (“AC”) current source, such as a radiofrequency (“RF”) current source; an antenna element which is energized by a microwave energy source; a heating element, such as a metallic element or other thermal conductor which is energized to emit heat such as by convection or conductive heat transfer, by resistive heating due to current flow, a light-emitting element (e.g., a laser), or an ultrasonic element such as an ultrasound crystal element which is adapted to emit ultrasonic sound waves sufficient to ablate a region of tissue when coupled to a suitable excitation source.

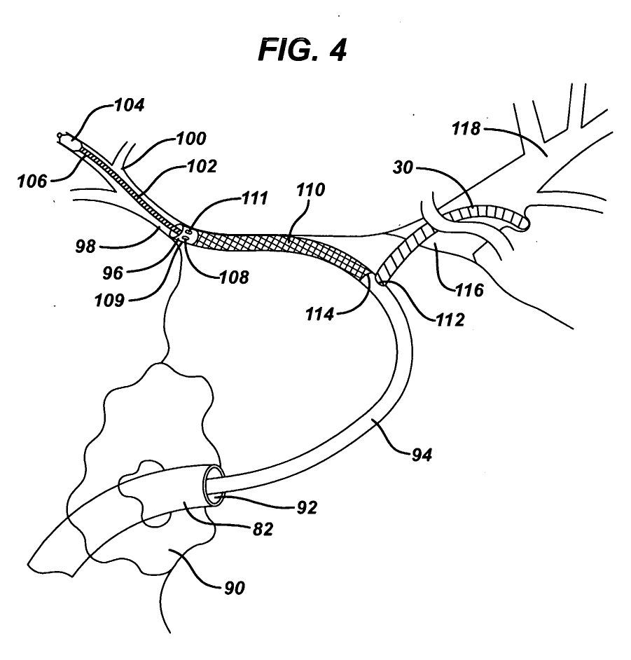

FIG. 4 shows another example of an ablation device, modified in accordance with the features of the present invention, in situ whereby a transeptal sheath 82 traverses the atrial septum 90 of the heart that separates the right and left atria. The distal end 92 of the transeptal sheath opens into the left atrium. Emerging from the transeptal sheath and slideably engaged therein is an ablation catheter 94. The ablation catheter 94 includes a light emission device 111 and light detection device 109. The distal end 96 of the ablation catheter 94 is shown engaging a region of tissue, for example, a first ostium 98, where the first pulmonary vein 100 extends from the atrium. A balloon anchor wire 102, having a balloon 104 on its distal end 106 is slideably engaged within the ablation catheter 94. The balloon 104 is located within the first pulmonary vein 100 and inflated so as to anchor the ablation catheter 94 in position within the first ostium 98 of the first pulmonary vein 100. Consequently, the distal end 108 of the linear ablation element 110 is secured at a location where the first pulmonary vein 100 extends from the atrium.

A deflectable guidewire 30 is shown emerging from the second guidewire port 112 in the ablation catheter 94. The deflectable guidewire 30 is slideably engaged within the ablation catheter 94 and the distal end 122 is adapted to be steerable by manipulating a pullwire (not shown) at the proximal end of the guidewire. Preferably, the deflectable guidewire 30 is advanced into the second pulmonary vein 118 and anchored therein by deflection of the distal end 122. By tracking over the deflectable guidewire 30, the proximal end 114 of the ablation element 110 can be positioned and secured at a location, for example, the second ostium 116, where the second pulmonary vein 118 extends from the atrium. The deflectable guidewire 30 may have been positioned within the second pulmonary vein using a preshaped guiding introducer as described above.

In operation, an ablation catheter is advanced into the targeted region where the lesion is to be formed, for example within the heart, liver or prostrate gland. The catheter is modified to include a light emitter that provides broadband and/or laser light to the lesion site. A light detector is also mounted on the ablation catheter to collect diffusely scattered illumination light. The ablation element of the catheter is energized whereby a lesion is formed in the surrounding tissue. Light from the emitter is scattered by the lesion. The light detector gathers and transmits the scattered light to a detection system. The detection system comprises a wavelength selective element that disperses the collected light into wavelengths of interest, and a quantification device.

The quantification device converts these measured light intensities into an electrical signal that can be processed with a computer and displayed graphically to the end-user of the ablation device. During surgical ablation, the operator obtains information about the lesion as it is being formed or, alternatively, can detect lesions that have already been formed. For example, the intensity of the scattered light changes due to ablation of tissue, allowing for an existing lesion to be located as the ablation catheter is advanced over tissue. Moreover, the depth of the lesion causes a corresponding change in the spectrum of scattered light. The operator can use this information to increase or decrease the energy delivered to the site varying the depth of the lesion or terminating the ablation procedure.

Although the present invention has been described above with respect to particular preferred embodiments, it will be apparent to those skilled in the art that numerous modifications and variations can be made to these designs without departing from the spirit or essential attributes of the present invention. Accordingly, reference should be made to the appended claims, rather than to the foregoing specification, as indicating the scope of the invention. The descriptions provided are for illustrative purposes and are not intended to limit the invention nor are they intended in any way to restrict the scope, field of use or constitute any manifest words of exclusion.

What is claimed is:

1. An apparatus comprising:
a means for altering structural or biochemical characteristics of a tissue site;
a means for emitting a bandwidth of electromagnetic energy towards the tissue site; and
a means for collecting and directing a bandwidth of scattered electromagnetic energy from the tissue site.

2. The apparatus of claim 1 wherein the means for altering structural or biochemical characteristics of tissue comprises a tissue ablation catheter.

3. The apparatus of claim 2 wherein the ablation catheter comprises an elongate body having an ablation element located at its distal end.

4. The apparatus of claim 3 wherein the ablation element emits energy such that the tissue site is altered when the ablation element is brought into contact therewith.

5. The apparatus of claim 4 wherein the elongate body is modified such that the emitting means is mounted therein whereby the tissue site is illuminated with a bandwidth of electromagnetic energy.

6. The apparatus of claim 5 wherein the elongate body is modified such that the collecting means is mounted therein whereby a bandwidth of scattered electromagnetic energy is received from the tissue site.

7. The apparatus of claim 1 wherein the means for emitting a bandwidth of electromagnetic energy comprises a fiber optic cable.

8. The apparatus of claim 1 wherein the means for emitting a bandwidth of electromagnetic energy comprises an LED.

9. The apparatus of claim 1 wherein the means for emitting a bandwidth of electromagnetic energy comprises a laser.

10. The apparatus of claim 1 wherein the means for collecting and directing a bandwidth of scattered electromagnetic energy comprises at least one lens.

11. The apparatus of claim 1 wherein the means for collecting and directing a bandwidth of scattered electromagnetic energy comprises at least one optical fiber.

12. The apparatus of claim 1 wherein the electromagnetic energy comprises light which illuminates said tissue site and is scattered thereby.

13. An apparatus comprising:
a flexible elongate body having a proximal end and a distal end;
an element configured on said distal end and adapted to alter structural or biochemical characteristics from a tissue site;
at least one first optical conduit adapted with said elongate substrate to direct a bandwidth of electromagnetic radiations at said tissue site; and
at least one second optical conduit adapted with said flexible elongate substrate to direct a received scattered bandwidth from said tissue site in order to real-time monitor and assess structural and/or biochemical characteristics from the tissue site.

14. The apparatus of claim 11 wherein said at least one first optical conduit is mounted within the elongate body near the distal end thereof.

15. The apparatus of claim 11 wherein said at least one second optical conduit is mounted within the elongate body near the distal end thereof.

16. The apparatus of claim 11 further comprising an electromagnetic radiation source for supplying a bandwidth of electromagnetic energy to the at least one first optical conduit.

17. The apparatus of claim 11 wherein the at least one second optical conduit receives a scattered bandwidth from said tissue site and directs it to a detection component which converts said scattered bandwidth into a digital signal.

18. The apparatus of claim 15 wherein the detection component comprises a device for dispersing the scattered bandwidth into constituent wavelengths, and a quantification device.

19. The apparatus of claim 16 wherein at least one wavelength selective element receives incident light and structures it into desired components that are transmitted into quantification apparatus.

20. The apparatus of claim 17 wherein the quantification device translates measured light intensities into an electrical signal that can be processed with a computer and displayed in a predetermined format.

21. The apparatus of claim 18 wherein the quantification device comprises a charged coupled device.

22. The apparatus of claim 18 wherein the quantification device a light sensor selected from a group consisting of photodiodes, photomultipliers and a complementary metal oxide semiconductor.
